(19) United States
(12) Patent Application Publication
(10) Pub. No.: US 2006/0161086 A1
(43) Pub. Date: Jul. 20, 2006

(54) Traction device

(76) Inventor: Dennis Lambert, Lincoln, CA (US)

Correspondence Address: JOHN P. O'BANION;O'BANION & RITCHEY LLP, 400 CAPITOL MALL SUITE 1550, SACRAMENTO, CA, 95814, US

(21) Appl. No.: 11/299,316

(22) Filed: Dec. 9, 2005

Related U.S. Application Data
(60) Provisional application No. 60/645,435, filed on Jan. 18, 2005.

(51) Int. Cl. A61F 5/00

(52) U.S. Cl. 602/32; 602/36

(57) ABSTRACT

An apparatus is shown for applying a traction force to a body member of a patient. The apparatus includes a columnar support coupling a lower arm to an upper arm. The columnar support has a fine adjustment mechanism for incrementally adjusting the distance between the lower arm and the upper arm along a central axis of the columnar support, wherein the distance corresponds to the traction force applied to the body member. The columnar support includes a plurality of sections that may be folded or separated for storage or transport. An attachment assembly, coupled to the upper arm, secures to one or more appendages in communication with the body member. A restraining element, coupled to the lower arm, secures to an opposing side of the body member such that the restraining element and the attachment assembly distribute the traction force to the body member upon actuation of the fine adjustment mechanism.

### CROSS-REFERENCE TO RELATED APPLICATIONS

This application claims priority from U.S. provisional application Ser. No. 60/645,435 filed on Jan. 18, 2005, incorporated herein by reference in its entirety. This application is also related to U.S. Pat. No. 6,811,541, also incorporated by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND OF THE INVENTION

1. Field of the Invention

This invention pertains generally to a traction apparatus, and more particularly to a portable traction apparatus.

2. Description of Related Art

Several traction devices exist, but each one fails to be of practical use in many real-life situations. As seen in U.S. Pat. No. 2,584,203, the traction apparatus uses rubber tubing to apply traction. The use of rubber tubing, under some situations, presents possible dangers to the patient and operator. Pulling tension with rubber tubing could potentially be hazardous due to a significant possibility of rubber break-down and separation, since the rubber could be ripped easily, develop holes, or could simply tear while in use. Replacement of torn rubber tubing takes time and is inconvenient. Further, this product is not portable in a user friendly way. Generally, this device either sits on the floor or on a table, thereby putting a patient several feet off the ground which is not ideal and usually impractical.

Disclosed in U.S. Pat. No. 5,074,291 is a device that involves a significant expense to fabricate. First, a user must purchase the related table and frame together. Second, this device may only be used in an operating room, since this invention was designed for surgery on the wrist or forearm. Many utilization options are precluded with this design.

U.S. Pat. No. 5,735,806 presents a wrist traction apparatus that it is not adjustable in many critical locations. Force is applied to both the thumb and a finger and if the operator wants to place added tension to the thumb, for alignment purposes, the finger next to the thumb experiences decreased tension. Thus, applying a deliberately stronger load on a specific finger is difficult, if not impossible, with the '806 apparatus. With the '806 tension apparatus the exact amount of force can not be determined. This could result is a possibly dangerous situation. Additionally, the traction pulley in the '806 apparatus is a relatively expensive “complete” component with no currently available parts for servicing.

Presented in U.S. Pat. No. 5,156,168 is a glove-like support for arthroscopy. Since arthroscopic type surgical procedures are only envisioned, in an operating room (OR) environment, this device would not work in a typical emergency room (ER) setting. The current subject apparatus may be utilized in both ER and OR settings and doesn't need a glove, which would need to be laundered.

Described in U.S. Pat. No. 4,445,506 is a bone aligning apparatus that is bulky and involves several time consuming fine-adjustments for use. Given the limited space available in a typical ER or OR environment, this apparatus would take up too much space and present a difficult cleaning problem. The fingers are placed into the finger holders, then an operator must manually adjust even tension once traction has begun which takes time. Further, with the '506 system there is no guarantee there will be an even distribution of traction to all of the secured fingers. The fixed position design not only restricts a patient's hand to that position only, which is not always needed, but the '506 device does not have individual finger adjustments so it limits the hand to solely that position.

U.S. Pat. No. 3,850,166 discloses a fracture reduction system that offers a wide range of configurations, but by presenting a wide range of configuration is overly complicated. With the '166 system there are a significant number of parts. If a part fails, it becomes complicated to replace them. Although the finger positioning can be adjusted manually, it does not allow for automatic finger tension adjustment simply by pulling traction. Additionally, the '166 device would be difficult to clean and sterilize.

U.S. Pat. Nos. 2,783,758 and 3,693,617 relate a fracture reducing device for the arm in which a table or table-like means supports the device. The arm is virtually fixed in a non-variable position and no automatic finger tension adjustment exists. Also, the traction device has only one finger trap, which could create undue stress on the finger (care must be taken of the associated ligaments and tendons in a hand and not to damage one by undue stress). Additionally, the necessity of having a sturdy table or table-like support limits the versatility of this devise, especially in spaces crowded with critical equipment. Further, in the '617 version a sandbag is utilized for downward force and is not adjustable.

### BRIEF SUMMARY OF THE INVENTION

An aspect of the invention is an apparatus for applying a traction force to a body member of a patient, comprising a columnar support coupling a lower arm to an upper arm. The columnar support has a fine adjustment mechanism for incrementally adjusting the distance between the lower arm and the upper arm along a central axis of the columnar support, wherein the distance corresponds to the traction force applied to the body member. The device further includes a base coupled to the columnar support for maintaining a substantially vertical orientation of the central axis. An attachment assembly is coupled to the upper arm, and is configured to secure to one or more appendages in communication with the body member. The traction force is applied to the body member through the one or more appendages such that the attachment assembly may selectively distribute the traction force applied to the one or more appendages. A restraining element is coupled to the lower arm, wherein the restraining element configured to be securably coupled to an opposing side of the body member from the one or more appendages such that the restraining element and the attachment assembly distribute the traction force to the body member upon actuation of the fine adjustment mechanism.

In one mode of the present aspect, the attachment assembly comprises a plurality of finger traps each adapted to retain a finger upon tensile loading of the finger trap. Each finger trap is releasably coupled to the upper arm via a spring, with a swivel base coupled between the spring and the upper arm.

In another mode, the apparatus further comprises a force measurement device, such as a scale, or strain guage, disposed between the attachment assembly and the upper arm.

In a preferred embodiment, the restraining element comprises an arm cuff configured to secure to the patient's arm.

In another mode, the upper arm, and/or the lower arm are configured to translate on the columnar support so as to provide course vertical adjustment of the distance between the upper arm and the lower arm. Additionally, the upper arm and/or lower arm may be configured to rotate about the columnar support so as to provide rotational adjustment of either the upper arm or lower arm about the central axis.

In another preferred embodiment, the columnar support comprises an extendable segment coupled to the fine adjustment mechanism such that the length of the extendable segment along the central axis may be varied upon actuation of the fine adjustment mechanism.

Preferably, the fine adjustment mechanism comprises a worm drive that allows for micro-motion of the extendable segment. For example, the fine adjustment mechanism may include an adjustment knob, wherein the worm drive converts rotational motion of the adjustment knob into linear motion of the extendable segment along the central axis.

In yet another embodiment, the columnar support is made up of a plurality of sections that allow the support to be broken down after use. The sections may be releasably secured, such as with a quick release mechanism, so that the section may be separated and reassembled quickly and easily.

In one variation of the current embodiment, the columnar support comprises an upper section supporting the upper arm, a lower section supporting the lower arm and an intermediate section linking the upper section to the lower section. Preferably, the extendable segment and the fine adjustment mechanism are housed in the intermediate section. However, these components may be housed in any section. Furthermore, it is appreciated that the columnar support comprise as little as one section, and up to any number of sections.

In another embodiment, the upper arm, lower arm and base all have hinged joints that allow them to collapse for storage. The columnar support may also have hinge joints, in place of, or in combination with separable sections.

Another aspect of the invention is an apparatus for applying a traction force to manipulate a body member in communication with a patient's arm, The apparatus has an extendable segment coupled to and separating an upper arm and a lower arm along a translation axis. A fine adjustment mechanism is coupled to the extendable segment for incrementally adjusting the distance between the lower arm and the upper arm along the translation axis. The upper arm is coupled to an attachment assembly configured to secure to one or more of the patient's fingers of the patients arm, and a restraining element configured to be securably coupled to the patient at an opposing location is coupled to the lower arm. Upon actuation of the fine adjustment mechanism, the extendable segment may be extended to generate the traction force applied to the body member through the patient's fingers and the opposing location via the attachment assembly and the restraining element, to manipulate said body member, wherein the attachment assembly is configured to selectively distribute the traction force applied through the one or more fingers.

In one mode of the present aspect, the attachment assembly comprises a plurality of finger traps each adapted to individually retain one of the fingers upon tensile loading of the finger trap, wherein each finger trap is releasably coupled to the upper arm via a spring. Each spring may be configured to equally or variably disperse the traction force among the one or more fingers.

Yet a further aspect of the invention is an apparatus for applying a traction force to manipulate a body member in communication with a patient's arm, comprising an extendable segment coupled to and separating the upper arm and the lower arm along a translation axis, and an adjustment means coupled to the extendable segment for incrementally adjusting the distance between the lower arm and the upper arm along the translation axis. The upper arm is coupled to an attachment means for securing the upper arm to one or more of the patient's fingers of the patient's arm, and a restraining means is coupled to the lower arm. Upon actuation of the fine adjustment means, the extendable segment may be lengthened to generate the traction force applied to the body member, wherein the traction force applied through the patient's fingers and an opposing location via the attachment means and the restraining means to manipulate said body member.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 illustrates an assembled traction device in accordance with the present invention.

FIG. 2 is a side view of an attachment assembly in accordance with the present invention.

FIG. 3A illustrates the finger traps of the present invention prior to attachment to the patient's hand.

FIG. 3B illustrates the finger traps of the present invention installed to the patient's hand.

FIG. 4 shows a bottom view of the swivel base in accordance with the present invention.

FIG. 5 illustrates an exemplary finger spacer in accordance with the present invention.

FIG. 6 illustrates a traction device in accordance with the present invention applied to a patient's arm.

FIG. 7 is a bottom view of the intermediate member of the present invention, illustrating a portion of the fine adjustment mechanism.

FIG. 8 illustrates the intermediate section in a separated configuration from the upper section, with the upper arm in a folded configuration.

FIG. 9 illustrates a top view of the base in a folded configuration.

### DETAILED DESCRIPTION OF THE INVENTION

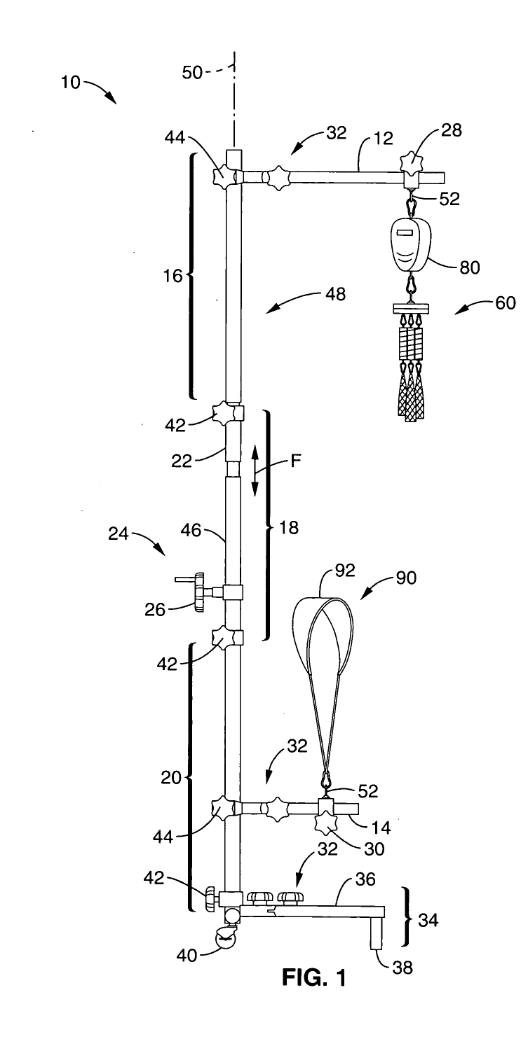

Referring more specifically to the drawings, for illustrative purposes the present invention is embodied in the apparatus generally shown in FIG. 1 through FIG. 9. It will be appreciated that the apparatus may vary as to configuration and as to details of the parts, and that the method may vary as to the specific steps and sequence, without departing from the basic concepts as disclosed herein.

FIG. 1 illustrates an exemplary traction device 10 in accordance with the present invention. Traction device 10 comprises an upper arm 12 and a lower arm 14 spaced vertically apart from each other along a columnar support 48. Columnar support 48 comprises three modular sections: upper section 16, intermediate section 18, and lower section 20, all of which generally lie along a central axis 50. Upper and lower arms 12,14 and columnar support sections 16,18, and 20 are generally tubular members comprised of a structurally rigid material, such as aluminum, steel, composite, or the like.

During use, the central axis 50 of the columnar support 48 is preferably retained in a vertical orientation via a base 34. Base 34 is releasably coupled to the lower section 20 via clamp 42. The base 34 has two horizontal members 36, each having a foot 38 at one end, wheel 40 at the other end. Wheels 40 allow traction device 10 to be moved along the ground without lifting the device. In an alternative embodiment (not shown), the base may have four wheel (without feet 38), and incorporate brakes, or similar mechanisms to keep the wheels from moving when not in transit.

The upper arm 12 is slideably received on upper section 16, and lower arm is slideably received on lower section 20 via releasable collar-type clamps 44. Clamps 44 may be tightened or loosened to allow adjustment of the upper arm 12 and lower arm 14 vertically along the central axis 50.

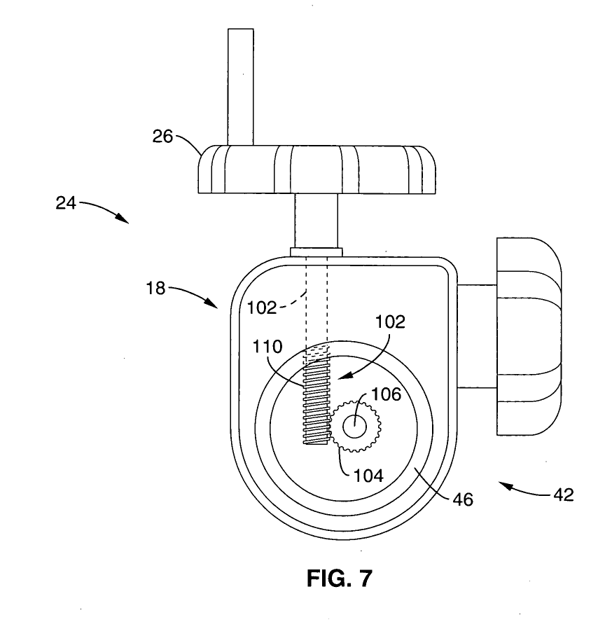

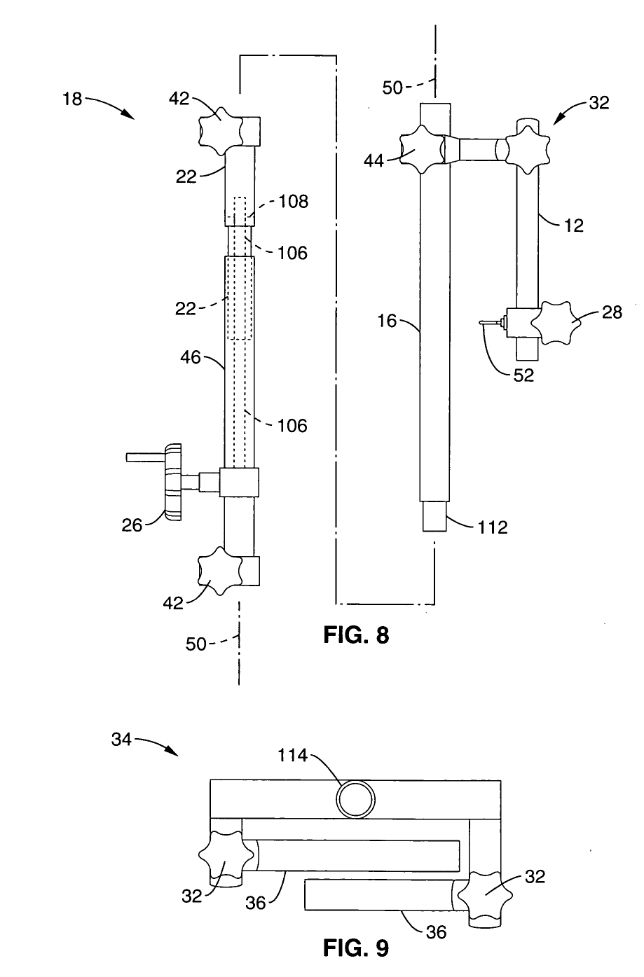

The intermediate section 18 has an extendable member 22 that is at least partially housed in stationary member 46 and coupled to a fine-adjustment mechanism 24. The adjustment mechanism 24 comprises a worm drive mechanism (explained in further detail with reference to FIGS. 7 and 8) that is coupled between an adjustment knob 26 and the extendable section 22. Hence, rotational motion of the adjustment knob 26 is converted to linear motion of the extendable member 22 with respect to the tubular stationary member 46. The translation of the extendable member 22 along the central axis acts to change the distance between the upper arm 12 and the lower arm 14. When driven to separate the upper arm and lower arm from each other, the adjustment mechanism 24 is used to impart a traction force on a body member of a patient.

The traction device 10 further comprises a first attachment means or assembly 60 for retaining a first aspect (e.g. one or more fingers of the patient's arm) of the patient's body member to be treated. The attachment assembly 60 is mechanically coupled to the upper arm 12 via eyelet 52 on slideable collar 28. Accordingly, the traction device 10 also has a second attachment means or assembly 90 for restraining a second aspect of the patient' body (e.g. patient's upper arm) to effect a traction force on the body member. The restraining element 90 is mechanically coupled to the lower arm 14 via eyelet 52 on slideable collar 30.

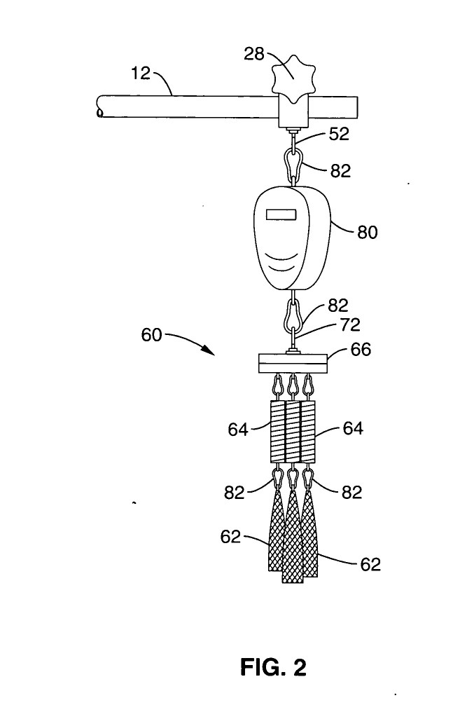

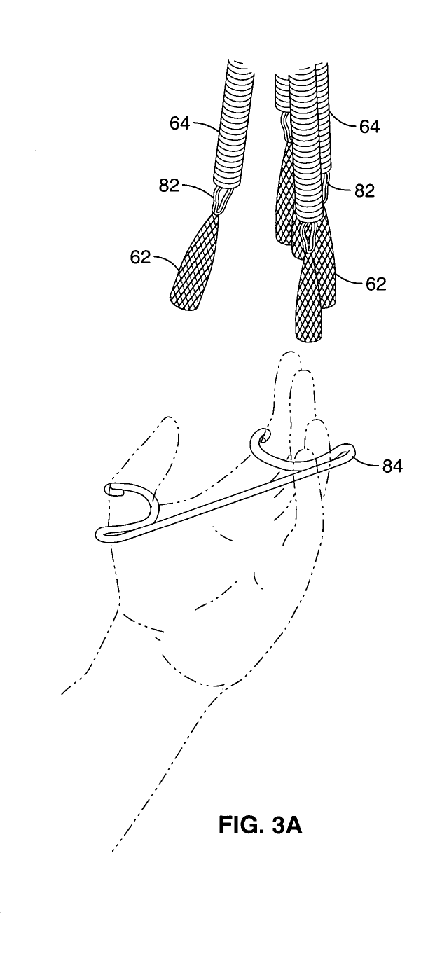

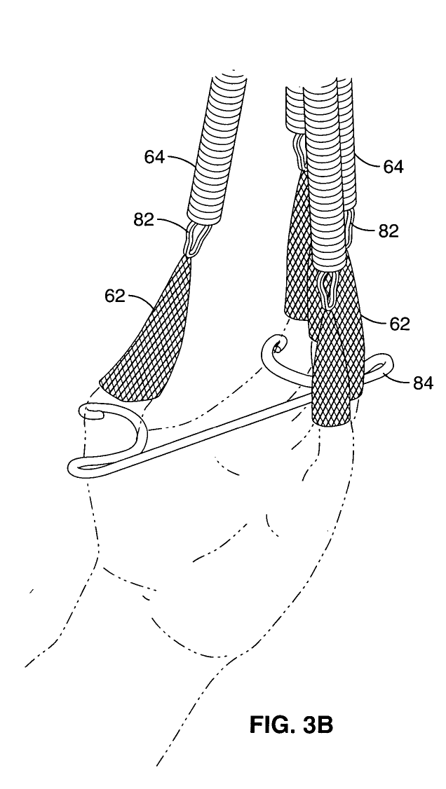

Attachment assembly 60 is further detailed in FIG. 2. A plurality of finger traps 62 are provided to secure the subject device to the patient. Each finger trap 62 preferably comprises finger cot having a tapered cylinder of interlaced filaments that reversibly grip or tighten on a finger placed within the trap 62 and pulled away from the opposite end. FIG. 2 illustrates an exemplary configuration of four finger traps. However, it is appreciated that as little as one, and as many as five, finger traps may be used. Each finger is released by relieving the tension on the trap 62 and working the filaments loose (FIGS. 3A and 3B show a patient's fingers free and inserted, respectively). One end of each trap 62 is wide and open to receive a finger and the other end narrows to a fastening means such as a ring, loop, or similar device.

The attachment assembly 60 further comprises a plurality of resilient means 64. Usually, each resilient means comprises a spring 64 or similar means such as an elastic member or the like. Each of the springs 64 is capable of releasably associating with at least one trap 62 by means of a releasable coupling such as a quick-release clip or carabineer 82. For desirable traction purposes, more than one spring, or a spring of differing resiliency, may be connected to any trap 62 to increase the traction force.

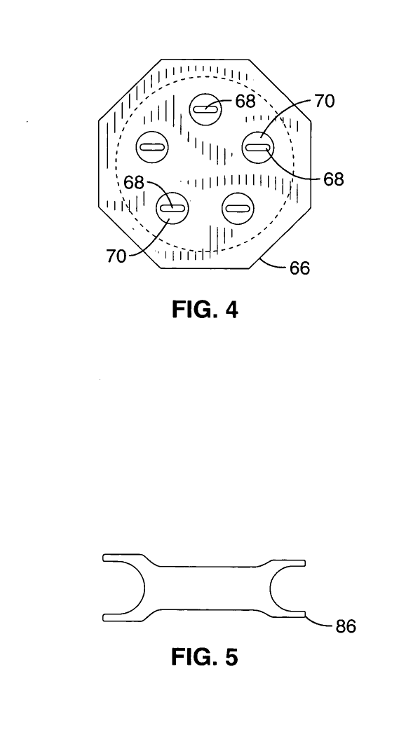

The attachment assembly 60 further comprises a swivel base 66 that secured to each of the springs 64. The swivel base 66 is secured to an upper end 28 of each spring 64 by suitable means. FIG. 4 depicts the bottom of the swivel base 66 and illustrates one suitable attachment means in which each spring end 68 fits within a receiving aperture 70 and is fastened in place by pinning means. Other suitable attachment means include, but are not limited to, quick-release clips, clamps, and the like. Preferably, to facilitate proper traction, the springs 64 are secured to the swivel base 66 in a generally circular attachment pattern (see FIG. 4). Included in and projecting from the swivel base 25 is a swiveling eyelet 72 that allows the attachment assembly 60 to swivel freely. The swivel eyelet 72 is usually an “eye” component (the “eye” presents a coupling aperture for securing to the remainder of the subject invention) rotationally secured in the swivel base 66.

The novel subject design of the attachment assembly 60 allows the traction device 10 to pull traction with exact tension to all fingers used, and also allows increase tension to any one digit safely and easily for situations when an operator desires to align certain bones by pulling on one finger slightly harder.

The attachment assembly 60 has a generally circular finger positioning design, along with the spring suspension system, to permit various hand alignment possibilities (utilizing a ball within the hand to generate a desired natural position). This advantageously allows for a natural position for the hand and hence easier alignment of bones, especially when the thumb is needed in the reduction. The subject spring suspension allows among other things, freedom to move fingers into positions needed for other concentrated reductions.

The current subject device is easy to use and self adjusts for tension and all parts are designed to rapidly disassemble. With the current subject device extra tension may be added to any finger just by a simple clip.

In a preferred embodiment, a force measurement device 80 is coupled between the attachment assembly 60 and the upper arm 12. Generally, carabineers 82 are used to couple both ends of the force measurement device 80 to the eyelets 52 and 72 of the upper arm 12 and attachment assembly 60. A suitable traction force measurement means 80 may comprise a strain gauge or an analog or digital scale or balance. For example, a 50 lb scale may be used to monitor the exact force applied to the patient's arm for traction purposes. It is appreciated that the force measurement device 80 may alternatively be coupled between the lower arm 14 and retaining member 90.

Referring now to FIGS. 3A and 3B, a finger spacer 84 may be utilized to position a patient's fingers during traction. FIG. 5 illustrates an alternative finger spacer 86 that may be used in place of spacer 84.

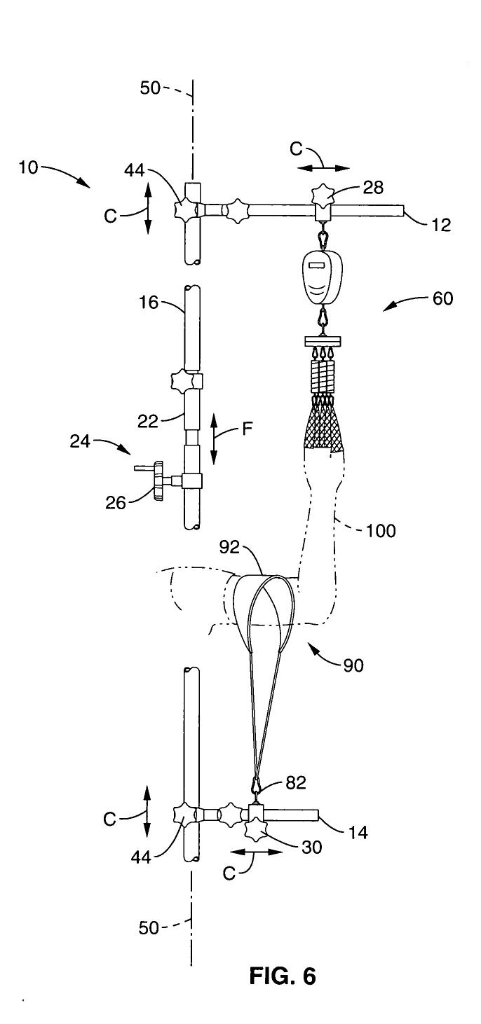

FIG. 6 illustrates the traction device 10 of the present invention being used to generate traction on region of a patient's arm 100. Prior to securing either attachment assembly 60 or 90 to the patient, the upper arm 12 and lower arm 14 may be coarsely adjusted (distance C) to the proper positioning according to the patients position (e.g. standing or seated) and anatomy. First the attachment points may be adjusted laterally (i.e. perpendicular to the central axis 50) by loosening collar 28 on the upper arm 12 and collar 30 on the lower arm 14. Once the proper lateral adjustment is made, the collar may be tightened to retain motion in the lateral direction.

Upper arm 12 and lower arm 14 may also be adjusted vertically along central axis 50 by loosening clamps 44. Rotation about the central axis 50 may also be facilitated at this time to angularly offset the lower arm 14 from the upper arm 12, if so desired. After the course adjustments are made, the claps 44 may be tightened to maintain the position of the upper and lower arms.

With the traction device properly positioned, the patient's fingers may then be secured to the attachment assembly 60 as shown in FIGS. 3A and 3B. The retaining member 90, which preferably comprises a padded arm cuff 95, may then be fit about the patient's upper arm. If necessary, further course adjustments may then be made by adjusting arms 12 and 14 via clamps 44 and collars 28 and 30.

Still referring to FIG. 6, traction force may then be applied to the patient's arm by rotating the adjustment knob 26 of the fine adjustment mechanism 24. The gearing on the worm drive is such that each rotation of the knob 26 only effects a small linear motion (e.g. micromotion) in the vertical direction (central axis). This motion separates the upper arm 12 from the lower arm 14 via extension of the extendable member 22. After any slack in the system is removed, further separating motion affects a tensile traction force F that is transmitted through the attachment assemblies 60 and 90, and into the patient's fingers and upper arm. The body member of interest (e.g. a fractured segment of the patient's wrist) is thus subject to the tensile force F, which may be incrementally increased upon further rotation of adjustment knob 26.

FIGS. 7 and 8 illustrate in further detail the fine adjustment mechanism 24. FIG. 7 shows a bottom view of the intermediate section 18. Adjustment knob 26 is rigidly coupled to worm 102, which extends horizontally into the interior cavity of the tubular stationary member 42. Worm 102 has a tooth 110 in the form of a screw thread that is configured to drive adjacent worm gear 104. Worm gear 104 has helical inclined teeth that are configured to engage the worm thread 110 while avoiding interference with worm 102. Gear 104 is positioned such that rotation of worm 102 causes a corresponding clockwise or counterclockwise rotation of worm gear 104. Worm gear 104 is rigidly coupled to vertical worm 106 such that rotation of worm gear 104 affects the same rotation of vertical worm 106.

The worm 102 drives the worm gear 104 and not vise versa. The mechanism locks if the worm gear 104 tries to drive the worm 102. Thus, after a traction force is generated on the body member, the loading is such that a force will be imparted to try to reverse the motion of the worm gear 104. However, since this motion is locked, the worm gear 104 will not back out, therefore maintaining the traction force until the adjustment knob 26 is moved. This configuration advantageously obviates the need for a ratchet or like mechanism, thus allowing for more precise adjustment with minimal or no slop or backlash. Worm drives also tend to run much quieter and smoother, a feature that can be much more appealing to a patient who is already in a traumatic predicament.

As shown in FIG. 8, vertical worm 106 runs generally parallel to, but offset from the central axis 50 along the length of the stationary member 46 and into the extendable member 22. Rigidly coupled to the extendable member 22 is a nut 108 (female helical threads) that is configured to interface with vertical worm 106 such that rotation of worm 106 advances the extendable member 22 with respect to the stationary member 46. The extendable member 22 is preferably keyed with respect to the stationary member such that the extendable member 22 is restricted from rotating along with worm 106. To lessen the traction force applied, or unload the body member, the extendable member 22 may be retracted by simply rotating the adjustment knob 26 in the opposite direction.

It is appreciated that other gearing mechanisms known in the art may be used in the fine adjustment mechanism 24 to drive the extendable member 22. However, the worm drive of the present invention is advantageous in that it allows easy rotational motion about an axis normal to the central axis 50 to affect a small, or micro motion, linearly along the central axis 50. Under the present gearing configuration, one revolution of the adjustment knob 26 results in 0.025 inches of linear travel of the extendable member 22 along the central axis relative to the stationary member 46. Thus, the adjustment knob 26 is turned 40 times for one linear inch of travel. This allows the operator to precisely, and incrementally, load the body member of interest.

It is appreciated that the worm drive may be geared accordingly to gain additional, or less precision. In the present embodiment, the worm drive has approximately 4 linear inches of travel, although it may be configured to have more or less based on the application.

Referring now to FIG. 8, the components of the traction device 10 are configured to disassemble and retract for storage and transport. FIG. 8 illustrates the intermediate section 18 and upper section 16, with the upper arm 12 folded for storage. To disassemble the upper section 16 from the intermediate section 18, the clamp 42 located on the extendable member 22 is rotated counter-clockwise and loosened. The upper section 18 may then be pulled out from the intermediate section 18 along central axis 50. The upper section 16 is generally a cylindrical tube that has a lower end 102 that has a smaller diameter than the rest of the tube, with the diameter of lower end 102 corresponding closely with the inner diameter of the receiving hole of extendable member 22. Locking the intermediate section 18 and upper section 16 together is facilitated by clockwise rotation of clamp 42 to tighten on the lower end 102 to the extendable member 22. The lower section 20, similarly interfaces with the clamp 42 on the stationary member 46.

It is appreciated that clamp 42, as well as collar clamps 44, may comprise a variety of configurations known in the art, such as a cam-type lock found on quick release skewers, or a depressible button type lock commonly used for interconnecting tubular members. Alternatively, the mating ends of each section may be threaded with corresponding male/female threads to releasably join each segment together.

FIG. 8 illustrates the upper arm 14 folded to run generally parallel to and adjacent to upper section 16. This is generally achieved by loosening joint 33 and rotating the upper arm 14 downward toward the upper section 16. Lower arm 14 (as shown in FIG. 1), may be similarly folded to run adjacent to lower section 20. This may be done while the device 10 is fully assembled so that the device 10 occupies less space in the treatment area. Alternatively, the arms 12 and 14 may be folded after all of the sectional members are separated for storage in a portable container.

FIG. 9 illustrates base 34 separated from the lower section 20 and in a folded configuration. As shown in FIG. 9, both of the horizontal members 36 may be folded inward after loosening releasable joints 32. The base is separated from the lower section 20 by loosening clamp 42 and sliding extension 114 (preferably having same diameter as and 112) from the inner diameter of the bottom end of the lower section 20.

It is appreciated that the columnar support 48 may comprise a number of configurations. For example, the support 48 may comprise a greater number of sections, such as five or more, to further compartmentalize the device for storage. Alternatively, the support may not be constructed of any separable sections, but rather comprise a plurality of foldable joints such as joints 32 of the upper and lower arms. A still further alternative is a support 48 having a combination of separable parts (e.g. separation between the base 34 and columnar support 48, and foldable joints disposed along the support.

Thus, the entire traction device 10 may be broken down and collapsed to be stored in a container for easy storage and transport. Accordingly, the device 10 may be reassembled in operating configuration quickly and easily. Thus, the compartmental nature of the present invention is highly advantageous in situations where the treatment room is an emergency room or the like, where space is limited and time is of the essence.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. An apparatus for applying a traction force to a body member of a patient, comprising:
an upper arm;
a lower arm;
a columnar support coupling the lower arm to the upper arm;
said columnar support having a central axis;
said columnar support further comprising a fine adjustment mechanism for incrementally adjusting the distance between the lower arm and the upper arm along the central axis;
said distance corresponding to the traction force applied to the body member;
an attachment assembly coupled to the upper arm;
the attachment assembly configured to secure to one or more appendages in communication with the body member;
said traction force being applied to said body member through said one or more appendages; and
a restraining element coupled to the lower arm, the restraining element configured to be securably coupled to an opposing side of the body member from the one or more appendages such that the restraining element and the attachment assembly distribute the traction force to the body member upon actuation of the fine adjustment mechanism.

2. An apparatus as recited in claim 1, further comprising:
a base coupled to the columnar support for maintaining a substantially vertical orientation of the central axis of the columnar support;

3. An apparatus as recited in claim 1:
wherein the attachment assembly is configured to selectively distribute the traction force applied to the one or more appendages.

4. An apparatus as recited in claim 3:
wherein the one or more appendages comprise one or more of the patient's fingers, and wherein the attachment assembly comprises:
a plurality of finger traps each adapted to retain a finger upon tensile loading of the finger trap;
each finger trap releasably coupled to the upper arm via a spring; and
a swivel base coupled between the spring and the upper arm.

5. An apparatus as recited in claim 1, further comprising:
a force measurement device disposed between the attachment assembly and the upper arm.

6. An apparatus as recited in claim 1, wherein the restraining element comprises an arm cuff configured to secure to the patient's arm.

7. An apparatus as recited in claim 1, wherein at least one of the upper arm and the lower arm are configured to translate on the columnar support so as to provide course vertical adjustment of the distance between the upper arm and the lower arm.

8. An apparatus as recited in claim 7, wherein at least one of the upper arm and the lower arm are configured to rotate about the columnar support so as to provide rotational adjustment of either the upper arm or lower arm about the central axis.

9. An apparatus as recited in claim 1:
wherein the columnar support comprises an extendable segment coupled to a stationary segment and the fine adjustment mechanism; and
wherein the length of the columnar support along the central axis may be varied upon actuation of the fine adjustment mechanism.

10. An apparatus as recited in claim 9, wherein the fine adjustment mechanism comprises a worm drive.

11. An apparatus as recited in claim 10:
wherein the fine adjustment mechanism further comprises an adjustment knob; and
wherein the worm drive converts rotational motion of the adjustment knob into linear motion of the extendable segment along the central axis.

12. An apparatus as recited in claim 9, wherein the columnar support comprises a plurality of sections that allow the support to be broken down after use.

13. An apparatus as recited in claim 12, wherein the columnar support comprises:
an upper section supporting the upper arm;
a lower section supporting the lower arm;
and an intermediate section linking the upper section to the lower section;
wherein the extendable segment and the fine adjustment mechanism are housed in the intermediate section.

14. An apparatus as recited in claim 13, wherein the upper section, lower section, intermediate section and base are fastened to each other via quick release mechanisms.

15. An apparatus as recited in claim 13, wherein the upper arm and lower arm have hinged joints that allow them to collapse for storage.

16. An apparatus for applying a traction force to manipulate a body member in communication with a patient's arm, comprising:
an upper arm;
a lower arm;
an extendable segment coupled to and separating the upper arm and the lower arm along a translation axis;
a fine adjustment mechanism coupled to the extendable segment for incrementally adjusting the distance between the lower arm and the upper arm along the translation axis;
wherein the upper arm is coupled to an attachment assembly configured to secure to one or more of the patient's fingers of the patients arm; and
a restraining element coupled to the lower arm, the restraining element configured to be securably coupled to the patient at an opposing location in communication with said body member;
wherein, upon actuation of the fine adjustment mechanism, the extendable segment may be extended to generate the traction force applied to the body member, said traction force applied through the patient's fingers and the opposing location via the attachment assembly and the restraining element, to manipulate said body member;
wherein the attachment assembly is configured to selectively distribute the traction force applied through the one or more fingers.

17. An apparatus as recited in claim 16, wherein the attachment assembly comprises:
a plurality of finger traps each adapted to individually retain one of the fingers upon tensile loading of the finger trap;
each finger trap releasably coupled to the upper arm via a spring; and
a swivel base coupled between the spring and the upper arm.

18. An apparatus as recited in claim 17, wherein each spring may be configured to equally disperse the traction force among the one or more fingers.

19. An apparatus as recited in claim 17, wherein each spring is configured to variably disperse the traction force among the one or more fingers.

20. An apparatus as recited in claim 16, wherein the restraining element comprises an arm cuff configured to secure to the patient's arm.

21. An apparatus as recited in claim 16, wherein the extendable segment is disposed within a columnar support;
the columnar support separating the upper arm from the lower arm.

22. An apparatus as recited in claim 21, wherein at least one of the upper arm and the lower arm are configured to translate on the columnar support so as to provide course vertical adjustment of the distance between the upper arm and the lower arm.

23. An apparatus as recited in claim 21, wherein at least one of the upper arm and the lower arm are configured to rotate about the columnar support so as to provide rotational adjustment of either the upper arm or lower arm about the central axis.

24. An apparatus as recited in claim 21, the fine adjustment mechanism comprising an adjustment knob coupled to a worm drive, wherein the worm drive converts rotational motion of the adjustment knob into linear motion of the extendable segment along the central axis to vary the distance between the upper arm and the lower arm.

25. An apparatus as recited in claim 21, wherein the columnar support comprises a plurality of sections that allow the support to be broken down after use.

26. An apparatus as recited in claim 21, wherein the columnar support comprises:
an upper section supporting the upper arm;
a lower section supporting the lower arm; and
an intermediate section linking the upper section to the lower section;
wherein the extendable segment and the fine adjustment mechanism are housed in the intermediate section.

27. An apparatus as recited in claim 26, wherein the upper section, lower section, intermediate section and base are fastened to each other via quick release mechanisms.

28. An apparatus as recited in claim 26, further comprising a base coupled to the lower section for maintaining a substantially vertical orientation of the central axis of the columnar support.

29. An apparatus as recited in claim 28, wherein the upper arm, lower arm and base all have hinged joints that allow them to collapse for storage.

30. An apparatus for applying a traction force to manipulate a body member in communication with a patient's arm, comprising:
an upper arm;
a lower arm;
an extendable segment coupled to and separating the upper arm and the lower arm along a translation axis;
an adjustment means coupled to the extendable segment for incrementally adjusting the distance between the lower arm and the upper arm along the translation axis;
wherein the upper arm is coupled to an attachment means for securing the upper arm to one or more of the patient's fingers of the patient's arm; and
a restraining means coupled to the lower arm, the restraining means for securably coupling the lower arm to the patient at an opposing location in communication with said body member;
wherein, upon actuation of the fine adjustment means, the extendable segment may be lengthened to generate the traction force applied to the body member, said traction force applied through the patient's fingers and the opposing location via the attachment means and the restraining means to manipulate said body member.

31. An apparatus as recited in claim 30, wherein the extendable segment is disposed on a columnar support.

32. An apparatus as recited in claim 31, wherein the columnar support comprises a plurality of sections releasably secured to each other by one or more fastening means;
said fastening means allowing the sections to be separated for storage of the apparatus.

33. An apparatus as recited in claim 30, wherein the attachment means is configured to selectively distribute the traction force applied through the one or more fingers.

34. An apparatus as recited in claim 30, wherein the adjustment means comprises a worm drive.
